(19) United States
(12) Patent Application Publication
(10) Pub. No.: US 2017/0056335 A1
(43) Pub. Date: Mar. 2, 2017

(54) Ophthalmic Composition

(71) Applicant: RHOTO PHARMACEUTICAL CO., LTD., Osaka (JP)

(72) Inventors: Masashi Itoh, Osaka (JP); Takenori Matsumoto, Osaka (JP); Chigusa Nakatsuka, Osaka (JP); Fumie Chono, Osaka (JP)

(73) Assignee: RHOTO PHARMACEUTICAL CO., LTD., Osaka (JP)

(21) Appl. No.: 15/302,636

(22) PCT Filed: Apr. 8, 2015
(86) PCT No.: PCT/JP2015/060989

§ 371 Date: Oct. 7, 2016

(30) Foreign Application Priority Data
Apr. 9, 2014 (JP) 2014-080315
May 29, 2014 (JP) 2014-110926
Sep. 5, 2014 (JP) 2014-181068
Oct. 3, 2014 (JP) 2014-204950

(51) Int. Cl. A61K 31/05; A61K 31/045; A61K 31/125; A61K 9/00; A61K 47/26

(52) U.S. Cl. CPC A61K 31/05; A61K 9/0048; A61K 47/26; A61K 31/125; A61K 31/045

(57) ABSTRACT

The present invention provides an ophthalmic composition comprising polysorbate 80 and a refreshing agent, wherein a polydispersity of the polysorbate 80 in the ophthalmic composition is 1.00 to 1.26.

### TECHNICAL FIELD

The present invention relates to ophthalmic compositions.

### BACKGROUND ART

The cornea is a tissue constructed at the outermost surface of the eyeball (the most exterior surface of the eyeball) and composed of 5 layers from the outside: the corneal epithelium, Bowman's membrane, corneal stroma, Descemet's membrane and corneal endothelium. Owing to the anatomical feature of being in direct contact with the outside, the cornea is susceptible to invasion by various foreign bodies such as antigens, microorganisms and dust. The corneal sensation is considered to be one of the sharpest sensations among pain sensations in the human body.

### CITATION LIST

### Patent Literature

[Patent Literature 1] Japanese Unexamined Patent Publication No. 2011-093889

### SUMMARY OF INVENTION

### Technical Problem

Invasion by foreign bodies such as dust and pollen to the corneal epithelium may cause such symptoms as a foreign body sensation or ocular pain. That is, it is important to prevent invasion by foreign bodies to the ocular surface and alleviate a foreign body sensation originating therefrom, in view of removing an unidentified complaint to enhance the quality of life.

As a measure against invasion by such foreign bodies, an eye wash which comprises a carboxyvinyl polymer and a monoterpene, where the amount used for each eye washing is 500 μL or more was found to remove a makeup or pollen from the eyeball or eyelid (Patent Literature 1).

The present invention has been made considering the above circumstances in order to provide an ophthalmic composition capable of increasing the inhibitory action on adsorption of foreign bodies to the cornea, to alleviate a foreign body sensation in the eye.

### Solution to Problem

The present inventors have conducted diligent research to solve the above problem, and as a result, newly confirmed that copresence of polysorbate 80 having a relatively high polydispersity in the ophthalmic composition and a refreshing agent inhibits the inhibitory effect on adsorption of foreign bodies to the cornea; however, copresence of polysorbate 80 having a relatively low polydispersity in the ophthalmic composition and a refreshing agent enhances the inhibitory effect on adsorption of foreign bodies to the cornea. That is, they found that an ophthalmic composition comprising (A) polysorbate 80 (hereinafter sometimes referred to as “Component (A)”) and (B) a refreshing agent (hereinafter sometimes referred to as “Component (B)”), wherein a polydispersity of Component (A) in the ophthalmic composition is 1.00 to 1.26, suppresses adsorption of foreign bodies to the cornea and relieves a foreign body sensation in the eye. The present inventors were the first to focus on the polydispersity of polysorbate 80 in an ophthalmic composition with respect to suppression on adsorption of foreign bodies to the cornea and an influence on a foreign body sensation in the eye. Based on these findings, the present inventors have completed the present invention.

Namely, the present invention provides ophthalmic compositions according to the following aspects. The details of the mechanism of suppression on adsorption of foreign bodies to the cornea or alleviation in a foreign body sensation in the eye with the polydispersity of polysorbate 80 set in the range below has not been elucidated, but it is believed as one reason that the micelle structure of the polysorbate 80 and the refreshing agent slightly differs depending on a difference in the polydispersity, affecting the affinity between corneal epithelial cells and foreign bodies.

Namely, the present invention provides the following [1] to [7] below.

[1] An ophthalmic composition comprising:

polysorbate 80, and

a refreshing agent,

wherein a polydispersity of the polysorbate 80 in the ophthalmic composition is 1.00 to 1.26, and

the refreshing agent comprises at least one selected from the group consisting of camphor, borneol and geraniol;

[2] The ophthalmic composition according to [1], wherein a total content of the refreshing agent is 0.00005 to 0.5 w/v % based on a total amount of the ophthalmic composition;
[3] The ophthalmic composition according to [1] or [2], wherein the total content of the refreshing agent is 0.0001 to 100 parts by mass with respect to 1 part by mass of a content of the polysorbate 80 contained in the ophthalmic composition;
[4] The ophthalmic composition according to any one of [1] to [3], wherein the ophthalmic composition is an eye-drops;
[5] The ophthalmic composition according to any one of [1] to [4], which is used for a contact lens;
[6] A reliever for a foreign body sensation, comprising the ophthalmic composition according to any one of [1] to [5];
[7] A method for imparting an action of relieving a foreign body sensation to an ophthalmic composition, comprising:

incorporating polysorbate 80 and a refreshing agent into the ophthalmic composition,

wherein a polydispersity of the polysorbate 80 in the ophthalmic composition is 1.00 to 1.26, and

the refreshing agent comprises at least one selected from the group consisting of camphor, borneol and geraniol.

Moreover, the present invention provides [P1] to [P7] below.

[P1] An ophthalmic composition comprising:

polysorbate 80, and

a refreshing agent,

wherein a polydispersity of the polysorbate 80 in the ophthalmic composition is 1.00 to 1.26;

[P2] The ophthalmic composition according to [P1], wherein the refreshing agent comprises at least one selected from the group consisting of menthol, camphor, borneol and geraniol;
[P3] The ophthalmic composition according to [P1] or [P2], wherein a total content of the refreshing agent is 0.0001 to 100 parts by mass with respect to 1 part by mass of a content of the polysorbate 80 contained in the ophthalmic composition;
[P4] The ophthalmic composition according to any one of [P1] to [P3], which is an eye-drops;
[P5] The ophthalmic composition according to any one of [P1] to [P4], which is for a soft contact lens;
[P6] A reliever for a foreign body sensation, comprising the ophthalmic composition according to any one of [P1] to [P5];
[P7] A method for imparting an action of relieving a foreign body sensation to an ophthalmic composition by incorporating polysorbate 80 and a refreshing agent therein,

wherein a polydispersity of the polysorbate 80 in the ophthalmic composition is 1.00 to 1.26.

In addition, the present invention provides [P8] to [P16] below.

[P8] A method for relieving a foreign body sensation in the eye, comprising:

a step of contacting an ophthalmic composition comprising polysorbate 80 and a refreshing agent with an eye or a contact lens of a subject,

wherein a polydispersity of the polysorbate 80 in the ophthalmic composition is 1.00 to 1.26;

[P9] The method according to [P8], wherein the refreshing agent comprises at least one selected from the group consisting of menthol, camphor, borneol and geraniol;
[P10] The method according to [P8] or [P9], wherein the subject is a human;
[P11] A use of polysorbate 80 and a refreshing agent for producing an ophthalmic composition for relieving a foreign body sensation in the eye,

wherein a polydispersity of the polysorbate 80 in the ophthalmic composition is 1.00 to 1.26;

[P12] Use of polysorbate 80 for producing an ophthalmic composition for relieving a foreign body sensation in the eye,

wherein the ophthalmic composition comprises a refreshing agent, and

a polydispersity of the polysorbate 80 in the ophthalmic composition is 1.00 to 1.26;

[P13] The use according to [P11] or [P12], wherein the refreshing agent comprises at least one selected from the group consisting of menthol, camphor, borneol and geraniol;
[P14] A refreshing agent and polysorbate 80 for use in relieving a foreign body sensation in the eye with an ophthalmic composition,

wherein a polydispersity of the polysorbate 80 in the ophthalmic composition is 1.00 to 1.26;

[P15] Polysorbate 80 for use in relieving a foreign body sensation in the eye with an ophthalmic composition,

wherein the ophthalmic composition comprises a refreshing agent, and

a polydispersity of the polysorbate 80 in the ophthalmic composition is 1.00 to 1.26;

[P16] The polysorbate 80 according to [P14] or [P15], wherein the refreshing agent comprises at least one selected from the group consisting of menthol, camphor, borneol and geraniol.

### Advantageous Effects of Invention

According to the present invention, an ophthalmic composition can be provided which is capable of increasing the inhibitory action on adsorption of foreign bodies to the cornea, and relieving a foreign body sensation in the eye. Specifically, an ophthalmic composition can be provided which reduces adsorption of foreign bodies such as dust, pollen, eye discharge, air pollution particles and makeup to the cornea, and relieves a foreign body sensation in the eye.

### DESCRIPTION OF EMBODIMENTS

Preferred embodiments of the present invention will be described in detail below. However, the present invention is not limited to the embodiments below. As used herein, the unit “%” of contents means “w/v %,” and has the same meaning with “g/100 mL.”

As used herein, a “foreign body sensation” means a subjective symptom of a gritty or stinging eye (a sense of strangeness, irritation) caused by adsorption of foreign bodies to the cornea. Such a subjective symptom is, for example, a subjective symptom feeling like something touches the eye when blinking, or a subjective symptom feeling like something is pricking the eye. Therefore, the present inventors focused on inhibition on adsorption of foreign bodies to the cornea in order to relieve a foreign body sensation in the eye, and alleviating subjective symptoms themselves. In particular, inhibition on adsorption of foreign bodies to the cornea shows a preventive effect on a foreign body sensation in the eye.

The ophthalmic composition according to this embodiment (hereinafter sometimes simply referred to as the “ophthalmic composition”) contains (A) polysorbate 80 and (B) a refreshing agent, where a polydispersity of the polysorbate 80 in the ophthalmic composition is 1.00 to 1.26, and the refreshing agent comprises at least one selected from the group consisting of camphor, bomeol and geraniol. An ophthalmic composition according to another embodiment contains (A) polysorbate 80, and (B) a refreshing agent, where a polydispersity of the polysorbate 80 in the ophthalmic composition is 1.00 to 1.26.

(Ophthalmic Composition)

Component (A)

The polysorbate 80 according to this embodiment (hereinafter sometimes simply referred to as “polysorbate 80”) is not particularly limited as long as its polydispersity is 1.00 to 1.26. The polysorbate 80 can be obtained by measuring polysorbate 80 obtained as a commercial product by the measurement method mentioned below, and selecting polysorbate 80 having a polydispersity of 1.00 to 1.26.

The polysorbate 80 having a polydispersity of 1.00 to 1.26 can be obtained by further fractionation or purification of polysorbate 80 obtained as a commercial product by a known method. The method for fractionating or purifying the polysorbate 80 is not particularly limited, but such a method as purification treatment using ion exchange resins, activated charcoal or adsorbents, steam deodorization treatment, decolorization treatment, dehydration and desolventization, filtration with a filter, cake filtration, centrifugation, sedimentation removal, or a method combining 2 or more of these can be used. These fractionation or purification methods can be performed when there is a need to remove an adverse effect on measurement errors of polydispersity or the like. Examples of other methods for fractioning the polysorbate 80 include such approaches as membrane filtration methods, approaches by distillation, extraction methods or washing with organic solvents, reprecipitation methods, liquid-liquid separation methods, or methods combining 2 or more of these. A specific example thereof is the following membrane filtration method. The molecular weight and polydispersity of polysorbate 80 obtained as a commercial product is measured by the size exclusion chromatography (SEC) method mentioned below, and a suitable ultrafiltration membrane is selected which has a rejection rate near 90% and provides a permeation flux. Commercially-available filtration membranes can be used (e.g., UF discs PLBC Ultracel RC 3K NMWL (molecular weight cutoff: 3 KDa, cellulose membrane) (manufactured by Millipore Corp.)). A pressure of 0.5 to 200 Pa is applied following the limit pressure of a filtration membrane. The polysorbate 80 having a polydispersity of 1.00 to 1.26 can be obtained by fractionation based on the weight average molecular weight and Z-average molecular weight.

Examples of other methods of purifying the polysorbate 80 include crystallization, resin chromatography, a method of drying and pulverizing the reaction solution as is with a spray dryer, a method of performing steam distillation for removal, and liquid-liquid extraction methods using organic solvents and water. A specific example of the above purification methods is the following method. First, polysorbate 80 is dissolved in methanol and measured for its molecular weight at the critical micelle concentration by a light scattering method, and the molecular weight cutoff of an ultrafiltration membrane is determined. Next, membrane filtration is performed at a condition of a flow rate of 0.5 to 10 ml/min, and a pressure of 0.1 to 20 bar.

The method of producing the polysorbate 80 is not particularly limited, but the polysorbate 80 having a polydispersity of 1.00 to 1.26 can be obtained by the following method. The production is conducted by adding ethylene oxide to a mixture of sorbitol and/or anhydrous sorbitol partially esterified with a fatty acid mainly consisting of oleic acid. The ester reaction can be performed by reacting sorbitan and the fatty acid under a basic catalyst, inert gas stream, and normal pressure or a reduced pressure, and at a temperature of commonly 150 to 280° C. After the reaction, a small amount of an acid enough to inactivate the basic catalyst is added for neutralization. The raw material sorbitol can be obtained by high-pressure hydrogen reduction of glucose, or commercially-available products may be used (examples thereof include Sorbit D-70 (manufactured by Towa Chemical Industry Co., Ltd.), Sorbitol S (manufactured by Nikken Chemicals Co., Ltd.)). Sorbitan can be obtained by dehydration cyclization of sorbitol, or commercially-available products may be used (examples thereof include those manufactured by Toei Chemical Co., Ltd.). Commercially available sorbitan oleate may also be used. The step of adding ethylene oxide to sorbitan fatty acid ester uses a catalyst such as alkali metal catalysts and fatty acid soaps. The produced polysorbate 80 can be filtrated. As the basic catalyst in the ester reaction, an alkaline compound such as alkali metal hydroxides such as sodium hydroxide, potassium hydroxide and lithium hydroxide, alkali metal carbonates such as sodium carbonate and potassium carbonate, alkali metal hydrogen carbonates, alkali metals, alkaline earth metals, alkaline earth metal oxides, alkaline earth metal hydroxides, other metals, their oxides, and mixtures thereof, and phosphorous acid (phosphite) and/or hypophosphorous acid (hypophosphite) are both used. As the inert gas used, nitrogen or argon can be used, for example. Examples of a metal catalyst for adding ethylene oxide to the product after the ester reaction include potassium hydroxide, sodium hydroxide, and sodium methylate. The polysorbate 80 having a polydispersity of 1.00 to 1.26 can be obtained by further fractionating or purifying the polysorbate 80 thus obtained by a known method.

Furthermore, the polysorbate 80 having a polydispersity of 1.00 to 1.26 may be prepared by blending 2 or more types of polysorbate 80 having different polydispersities.

The ophthalmic composition according to this embodiment may be produced by a method including the step of incorporating the polysorbate 80 having a polydispersity of 1.00 to 1.26 obtained as a commercial product, the polysorbate 80 having a polydispersity of 1.00 to 1.26 obtained by purifying a commercial product by the above purification method, or the polysorbate 80 having a polydispersity of 1.00 to 1.26 produced by the above production method, and a refreshing agent. The ophthalmic composition according to this embodiment may be produced by a method including the step of incorporating 2 or more types of polysorbate 80 obtained by different ways, and a refreshing agent. Production by such a method enables the polydispersity of polysorbate 80 in the ophthalmic composition to be 1.00 to 1.26.

The polydispersity of polysorbate 80 is 1.00 to 1.26, and preferably 1.00 to 1.23, more preferably 1.00 to 1.21, and still more preferably 1.00 to 1.19. When the polydispersity is in these ranges, the effects of the present invention—inhibition on adsorption of foreign bodies to the cornea or alleviation of a foreign body sensation in the eye—can be more significantly exhibited.

The weight average molecular weight of the polysorbate 80 is not particularly limited, but preferably in the range of 1200 to 3200, and more preferably in the range of 1300 to 3000. The Z-average molecular weight of the polysorbate 80 is not particularly limited, but preferably in the range of 1300 to 3500, and more preferably in the range of 1400 to 3300.

When the weight average molecular weight, and Z-average molecular weight of the polysorbate 80 is in the ranges as mentioned above, the effects of the present invention—inhibition on adsorption of foreign bodies to the cornea or alleviation of a foreign body sensation in the eye—can be more significantly exhibited.

The methods of measuring and calculating the weight average molecular weight, Z-average molecular weight and polydispersity are as mentioned below.

The polydispersity of polysorbate 80 is defined as M_{z}/M_{w}. M_{z} represents the Z-average molecular weight, and M_{w} represents the weight average molecular weight.

Various methods of measuring molecular weight are known such as liquid chromatography, ultracentrifugation methods, light scattering methods and intrinsic viscosity methods. Different measurement methods provide different types of average molecular weight obtained such as weight average molecular weight or Z-average molecular weight, or measurable molecular weight ranges. The weight average molecular weight, Z-average molecular weight and polydispersity as defined in the invention of the present application are measured by a size exclusion chromatography (SEC) method. SEC methods can measure weight average molecular weight, Z-average molecular weight and polydispersity, and are easily operable, and thus are the most widely used methods for measuring macromolecular compounds, oligomers or the like.

Using a column filled with a porous gel, SEC methods have a mechanism of separation by differences in elution time due to differences in migration distance in the column depending on the molecular sizes of molecules in a sample solution. Average molecular weights such as M_{z} and M_{w} are calculated based on a calibration curve created by a standard substance of known molecular weight. Thus, the Z-average molecular weight, weight average molecular weight and polydispersity obtained by SEC methods are relative values obtained by conducting molecular weight calibration using a standard substance, and therefore they are obtained only when the same standard substance is used. Then, the SEC method of the invention of the present application is defined as using polyethylene oxide and polyethylene glycol as the standard substance as described below. The Z-average molecular weight, weight average molecular weight and polydispersity obtained by SEC methods are relative values, and thus condition setting is also important in separation of components to be detected and creation of the calibration curve. The present inventors intensively studied the measurement conditions for the SEC method in order to increase the accuracy of calculation of the Z-average molecular weight, weight average molecular weight and polydispersity of the polysorbate 80 considering these characteristics of the SEC method.

In this embodiment, the weight average molecular weight, Z-average molecular weight and polydispersity of the polysorbate 80 in the ophthalmic composition is measured as follows.

### (Preparation of Calibration Solution)

1. Polyethylene oxides (Tosoh Corporation) and polyethylene glycols (Wako Pure Chemical Industries, Ltd.) having molecular weights from several hundred thousand to several hundred are used as the standard substance. It is preferred that polyethylene oxides and polyethylene glycols used as the standard substance are 3 types having molecular weights of several hundred thousands, 2 types having molecular weights of several ten thousands, 2 types having molecular weights of several thousands, and 1 type having a molecular weight of several hundreds. Further preferably, polyethylene oxides having molecular weights of 580000, 255000, 146000, 44900, and 27000 respectively, as well as polyethylene glycols having molecular weights of 8000, 1000, and 600 respectively are used. They are divided into combinations: those having molecular weights of 580000, 146000, 27000, and 1000 respectively, and those having molecular weights of 255000, 44900, 8000, and 600 respectively, so that elution positions do not overlap.
2. A standard solution is made by preparing an aqueous solution having a concentration of the standard substance of 0.1 w/v %. It is very important that the concentration of the standard substance be 0.1 w/v %, in order to suppress relative standard deviation in the Z-average molecular weight, weight average molecular weight, and polydispersity.
3. A calibration solution is made by taking 1.0 mL of the standard solution precisely, and adding 4.0 mL of methanol to the solution.

### (Preparation of Sample Solution)

4. A sample solution is made by taking 1.0 mL of a test solution precisely, and adding 4.0 mL of methanol to the solution.

### (Measurement by Liquid Chromatograph)

5. Calibration solutions and sample solutions are each measured with a liquid chromatograph (Agilent 1200 Series, manufactured by Agilent Technologies).
6. The columns are from Tosoh Corporation, and a column of TSK-Gela-4000 (exclusion limit molecular weight of about 400000) and a column of TSK-Gela-2500 (exclusion limit molecular weight of about 5000) (inner diameter: 7.8 mm, length: 300 mm, both columns), both packed with a hydrophilic vinyl polymer are connected and set up in this order. The column temperature is 40.0° C. It is very important to keep 40.0° C. constant in order to suppress relative standard deviation of the Z-average molecular weight, weight average molecular weight, and polydispersity.
7. As the eluent, a mixed solution containing 0.10 mol/L aqueous sodium chloride and 4.0 times its volume of methanol is prepared for each measurement and used. It is very important that the eluent contain sodium chloride to suppress interactions between sample molecules or between sample molecule and a packing material, for suppressing relative standard deviation of the Z-average molecular weight, weight average molecular weight, and polydispersity.
8. The flow rate is 0.5 mL/min.
9. The injection amount is 50 μL. Before injection, filtration with a 0.45 μm filter is conducted.
10. The calibration solution, and sample solution are measured in this order, and the analysis cycle for the calibration solution is 60 minutes, the sample solution 90 minutes. It is very important to continuously measure the calibration solution and the sample solution without interrupting the measurement, in order to suppress relative standard deviation of the Z-average molecular weight, weight average molecular weight, and polydispersity.
11. The detector is a differential refractive index detector, and the detector temperature is 35.0° C.

### (Calculation of the Z-Average Molecular Weight, Weight Average Molecular Weight and Polydispersity)

12. The obtained chromatogram is analyzed by a SEC analyzer, and the Z-average molecular weight and weight average molecular weight are calculated for the range between the chromatogram curve and the baseline. The elution time and value of molecular weight of each peak from the calibration solutions are plotted, approximation is made by a linear expression to make a calibration curve, and the correlation coefficient is 0.99 or more. Furthermore, to reduce errors in chromatogram analysis, the obtained peak is vertically partitioned in intervals of 0.1 minute or more and less than 0.001 minute, and the average molecular weight is calculated.
13. The polydispersity is calculated by the expression M_{z}/M_{w}. The sample solution of the step shown in 4. above is measured in triplicate, and average values of the Z-average molecular weights, weight average molecular weights or polydispersities obtained each time are employed as the Z-average molecular weight, weight average molecular weight or polydispersity of the present application, respectively.

The relative standard deviation of average molecular weight in SEC methods generally known is calculated by the following Expression A and Expression B, and is about 4 to 5%. In the above-mentioned SEC measurement condition, the relative standard deviation of polydispersity is less than 5%, and thus it can be considered as an appropriate measurement condition. The polydispersity in the present application is measured and calculated so that the relative standard deviation be less than 5%. When the above-mentioned reagent, column, or liquid chromatograph is not available, substitutes equivalent to these can be used to conduct the measurement. However, the measurement needs to be conducted so that the relative standard deviation of polydispersity be less than 5%. When the relative standard deviation is 5% or more, there is a need to suppress the relative standard deviation in a range studied of measurement conditions commonly practiced. In the study of the present inventors, it was important to satisfy the above condition with respect to the concentration of the standard substance, the column temperature, the composition of the eluent, and the analysis cycle of the calibration solution and sample solution, for suppressing the relative standard deviation.

$$\begin{matrix} Standard deviation S . D . = \sqrt{\frac{\sum {( Xi - \overline{X} )}^{2}}{n - 1}} & Expression A \\ Relative standard deviation C . V . ( \% ) = ( \frac{\begin{matrix}Standard \\ deviation\end{matrix}}{\overline{X}} ) \times 100 & Expression B\end{matrix}$$

In the ophthalmic composition according to this embodiment, the content of Component (A) is not particularly limited, and set as appropriate depending on the type and content of Component (B) used in combination. The content of Component (A) is, for example, based on the total amount of the ophthalmic composition, preferably 0.0001 to 8 w/v %, more preferably 0.0001 to 4 w/v %, further preferably 0.0005 to 2 w/v %, and particularly preferably 0.01 to 1 w/v %. The above content of Component (A) is preferred in view of the effects of inhibition on adsorption of foreign bodies to the cornea or alleviation of a foreign body sensation in the eye.

Component (B)

Component (B) is a refreshing agent. As Component (B), one type may be used singly, or two or more types may be used in any combination.

The refreshing agent used in the ophthalmic composition according to this embodiment is not particularly limited as long as it is medicinally, phatinacologically (pharmaceutically) or physiologically acceptable. Examples of the refreshing agent include terpenoids, essential oils containing terpenoids (e.g., eucalyptus oil, bergamot oil, peppermint oil, fennel oil, rose oil, cinnamon oil, spearmint oil, camphor oil, cool mint and mint oil). Examples of the terpenoid include menthol, menthone, camphor, borneol (also referred to as “camphol” or “Borneo camphor”), geraniol, nerol, cineol, citronellol, carvone, anethole, eugenol, limonene, linalool and linalyl acetate. The terpenoid may be any of d-isomers, 1-isomers and dl-isomers, and examples thereof include 1-menthol, d-menthol, dl-menthol, di-camphor, d-camphor, dl-borneol and d-borneol. However, like geraniol, nerol, cineol or the like, some terpenoids do not have optical isomers. In view of the effects of inhibition on adsorption of foreign bodies to the cornea or alleviation of a foreign body sensation in the eye, it is preferable that the refreshing agent comprise at least one selected from the group consisting of menthol, camphor, borneol and geraniol, and it is particularly preferable that the refreshing agent comprise at least one selected from the group consisting of 1-menthol, dl-camphor, d-camphor and d-borneol.

The content of the refreshing agent in the ophthalmic composition according to this embodiment is not particularly limited, and set as appropriate depending on the type of the refreshing agent, the type and content of Component (A) used in combination or the like. The content of the refreshing agent can be measured as terpenoids, and for example, based on the total amount of the ophthalmic composition, the total content of the refreshing agent (as terpenoids) is preferably 0.00005 to 0.5 w/v %, more preferably 0.0001 to 0.3 w/v %, further preferably 0.0005 to 0.2 w/v %, and particularly preferably 0.001 to 0.1 w/v %. The above content of the refreshing agent is preferable in view of the effects of inhibition on adsorption of foreign bodies to the cornea or alleviation of a foreign body sensation in the eye.

The content ratio of the refreshing agent to Component (A) in the ophthalmic composition according to this embodiment is not particularly limited, and set as appropriate depending on the type of the refreshing agent, etc. As for the content ratio of the refreshing agent to Component (A), for example, the total content of the refreshing agent is preferably 0.0001 to 100 parts by mass, more preferably 0.001 to 80 parts by mass, further preferably 0.005 to 60 parts by mass, and particularly preferably 0.01 to 50 parts by mass, with respect to 1 part by mass of the content of Component (A) contained in the ophthalmic composition. The above content ratio of the refreshing agent to Component (A) is preferable in view of the effects of inhibition on adsorption of foreign bodies to the cornea or alleviation of a foreign body sensation in the eye.

It is preferred that the ophthalmic composition according to this embodiment further contain a buffering agent. This enables adjustment of the pH of the ophthalmic composition, and the effects of the present invention can be more significantly exhibited.

The buffering agent is not particularly limited as long as it is medicinally, pharmacologically (pharmaceutically) or physiologically acceptable. Examples of the buffering agent include borate buffering agents, phosphate buffering agents, carbonate buffering agents, citrate buffering agents, acetate buffering agents, tris buffering agents, aspartic acid, aspartates, and edetates. These buffering agents may be used singly, or in any combination of two or more. Examples of borate buffering agents include boric acid or its salts (alkali metal salts of boric acid, alkaline earth metal salts of boric acid, or the like). Examples of phosphate buffering agents include phosphoric acid or its salts (alkali metal salts of phosphoric acid, alkaline earth metal salts of phosphoric acid, or the like). Examples of carbonate buffering agents include carbonic acid or its salts (alkali metal salts of carbonic acid, alkaline earth metal salts of carbonic acid, or the like). Examples of citrate buffering agents include citric acid or its salts (alkali metal salts of citric acid, alkaline earth metal salts of citric acid, or the like). As the borate buffering agent or phosphate buffering agent, a hydrate of a borate or phosphate may be used. By way of more specific example, examples of borate buffering agents include boric acid or its salts (sodium borate, potassium tetraborate, potassium metaborate, ammonium borate, borax or the like); examples of phosphate buffering agents include phosphoric acid or its salts (disodium hydrogen phosphate, sodium dihydrogen phosphate, potassium dihydrogen phosphate, trisodium phosphate, tripotassium phosphate, calcium monohydrogen phosphate, calcium dihydrogen phosphate or the like); examples of carbonate buffering agents include carbonic acid or its salts (sodium hydrogen carbonate, sodium carbonate, ammonium carbonate, potassium carbonate, calcium carbonate, potassium hydrogen carbonate, magnesium carbonate or the like); examples of citrate buffering agents include citric acid or its salts (sodium citrate, potassium citrate, calcium citrate, sodium dihydrogen citrate, disodium citrate or the like); examples of acetate buffering agents include acetic acid or its salts (ammonium acetate, potassium acetate, calcium acetate, sodium acetate or the like); aspartic acid or its salts (sodium aspartate, magnesium aspartate, potassium aspartate or the like). Among these buffering agents, borate buffering agents (e.g., a combination of boric acid and borax or the like) or phosphate buffering agents (e.g., a combination of disodium hydrogen phosphate and sodium dihydrogen phosphate or the like) are preferred, and borate buffering agents are further preferred.

When the buffering agent is incorporated in the ophthalmic composition according to this embodiment, its content is set as appropriate depending on the type of the buffering agent, the types and contents of other components contained, the application, product form, usage of the ophthalmic composition, etc. As for the content of the buffering agent, for example, based on the total amount of the ophthalmic composition, the total content of the buffering agent is preferably 0.001 to 15 w/v %, more preferably 0.01 to 10 w/v %, further preferably 0.05 to 7.5 w/v %, and particularly preferably 0.1 to 5 w/v %.

It is preferred that the ophthalmic composition according to this embodiment further contain a chelating agent. This enables stable long-term use of the ophthalmic composition, and the effects of the present invention can be more significantly exhibited.

The chelating agent is not particularly limited as long as it is medicinally, pharmacologically (pharmaceutically) or physiologically acceptable. Examples of the chelating agent include ethylenediaminediacetic acid (EDDA), ethylenediaminetriacetic acid, ethylenediaminetetraacetic acid (edetic acid, EDTA), N-(2-hydroxyethyl)ethylenediaminetriacetic acid (HEDTA), diethylenetriaminepentaacetic acid (DTPA). Among these chelating agents, ethylenediaminetetraacetic acid is preferred. These chelating agents may be used singly, or in any combination of two or more.

When the chelating agent is incorporated in the ophthalmic composition according to this embodiment, its content is set as appropriate depending on the type of the chelating agent, the types and contents of other components contained, the application, product form and usage of the ophthalmic composition, etc. As for the content of the chelating agent, for example, based on the total amount of the ophthalmic composition, the total content of the chelating agent is preferably 0.0001 to 2 w/v %, more preferably 0.0004 to 1.5 w/v %, further preferably 0.0008 to 1 w/v %, and particularly preferably 0.001 to 0.8 w/v %.

The pH of the ophthalmic composition according to this embodiment is not particularly limited as long as it is within a medicinally, pharmacologically (pharmaceutically) or physiologically acceptable range. The pH of the ophthalmic composition is, for example, in the range of 4.0 to 9.5, preferably in the range of 4.5 to 9.0, and more preferably in the range of 5.0 to 8.5. When the pH is in the above ranges, irritation to the eye is less when using the ophthalmic composition, adsorption of foreign bodies to the cornea tends to be further inhibited, and a foreign body sensation in the eye tends to be more alleviated.

It is preferred that the ophthalmic composition according to this embodiment further contain a tonicity agent. This enables adjustment of the osmotic pressure of the ophthalmic composition, and the effects of the present invention can be more significantly exhibited. The tonicity agent is not particularly limited as long as it is medicinally, pharmacologically (pharmaceutically) or physiologically acceptable. Examples of the tonicity agent include disodium hydrogen phosphate, sodium dihydrogen phosphate, potassium dihydrogen phosphate, sodium bisulfate, sodium sulfite, potassium chloride, calcium chloride, sodium chloride, magnesium chloride, potassium acetate, sodium acetate, sodium hydrogen carbonate, sodium carbonate, sodium thiosulfate, magnesium sulfate, glycerin, propylene glycol, polyethylene glycol, glucose, mannitol, sorbitol. Among these tonicity agents, glycerin, propylene glycol, polyethylene glycol, glucose, sodium chloride, potassium chloride, calcium chloride or magnesium chloride are preferred, sodium chloride, potassium chloride or propylene glycol are further preferred, and sodium chloride is particularly preferred. These tonicity agents may be used singly, or in any combination of two or more.

When the ophthalmic composition according to this embodiment contains a tonicity agent, its content is set as appropriate depending on the type of tonicity agent, the types and contents of other components contained, etc. As for the content of the tonicity agent, for example, based on the total amount of the ophthalmic composition, the total content of the tonicity agent is preferably 0.001 to 10 w/v %, more preferably 0.01 to 5 w/v %, and further preferably 0.05 to 3 w/v %.

The osmotic pressure of the ophthalmic composition according to this embodiment is not particularly limited as long as it is within a range acceptable for living body. The osmolar ratio of the ophthalmic composition is, for example, preferably 0.5 to 5.0, more preferably 0.6 to 3.0, further preferably 0.7 to 2.0, and particularly preferably 0.8 to 1.6. The adjustment of the osmotic pressure can be performed by a method known in the art using an inorganic salt, polyhydric alcohol, sugar alcohol or sugar. The osmolar ratio is a ratio of the osmotic pressure of a sample to 286 mOsm (the osmotic pressure of 0.9 w/v % aqueous sodium chloride) based on the Japanese Pharmacopoeia, Sixteenth Edition, and the osmotic pressure is measured with reference to Osmolarity Determination (freezing-point depression method) described in the Japanese Pharmacopoeia. The standard solution for osmolar ratio determination (0.9 w/v % aqueous sodium chloride) may be prepared by weighing exactly 0.900 g of sodium chloride (the Japanese Pharmacopoeia standard reagent), previously dried between 500 and 650° C. for 40 to 50 minutes and allowed to cool in a desiccator (silica gel), and dissolving the weighed sodium chloride in purified water to make exactly 100 mL of the solution, or commercially-available standard solutions for osmolar ratio determination (0.9 w/v % aqueous sodium chloride) may be used.

It is preferred that the ophthalmic composition according to this embodiment further contain a thickening agent. This enables adjustment of the viscosity of the ophthalmic composition, and the effects of the present application can be more significantly exhibited.

Examples of the thickening agent include polyvinyl alcohols (fully saponified or partially saponified), polyvinylpyrrolidones (K25, K30, K90, etc.), carboxyvinyl polymers, cellulose derivatives [methyl cellulose, ethyl cellulose, hydroxyethyl cellulose, hydroxypropyl cellulose, hydroxypropyl methylcellulose (2208, 2906, 2910, etc.), carboxymethyl cellulose, carboxyethyl cellulose, nitrocellulose or their salts and the like], polyethylene glycols (macrogol 300, macrogol 400, macrogol 1500, macrogol 4000, macrogol 6000, etc.), sodium chondroitin sulfate, sodium hyaluronate, gum arabic, gellan gum, tragacanth, dextran (40, 70, etc.), glucose, sorbitol; and the thickening agent is preferably polyvinyl alcohols (fully saponified or partially saponified), polyvinylpyrrolidones (K25, K30, K90), carboxyvinyl polymers, cellulose derivatives [methyl cellulose, hydroxyethyl cellulose, hydroxypropyl cellulose, hydroxypropyl methylcellulose (2208, 2906, 2910), carboxymethyl cellulose or their salts and the like], polyethylene glycols (macrogol 300, macrogol 400, macrogol 4000, macrogol 6000, etc.) or dextran (70), and further preferably polyvinyl alcohols (fully saponified or partially saponified), polyvinylpyrrolidones (K25, K30, K90), carboxyvinyl polymers, methyl cellulose, hydroxyethyl cellulose, hydroxypropyl methylcellulose (2208, 2906, 2910), carboxymethyl cellulose or salts thereof, polyethylene glycols (macrogol 300, macrogol 400, macrogol 4000, macrogol 6000) or dextran (70). In view of the effects of inhibition on adsorption of foreign bodies to the cornea or alleviation of a foreign body sensation in the eye, polyvinylpyrrolidones, hydroxyethyl cellulose or hydroxypropyl methylcellulose are further preferable, and polyvinylpyrrolidones are particularly preferable. These thickening agents may be used singly, or in any combination of two or more.

When the ophthalmic composition according to this embodiment contains a thickening agent, its content is set as appropriate depending on the type of thickening agent, the types and contents of other components contained, etc. As for the content of the thickening agent, for example, based on the total amount of the ophthalmic composition, the total content of the thickening agent is preferably 0.0001 to 5 w/v %, more preferably 0.0005 to 3 w/v %, further preferably 0.001 to 2 w/v %, and particularly preferably 0.01 to 1 w/v %.

The viscosity of the ophthalmic composition according to this embodiment is not particularly limited as long as it is within a range acceptable for living body. The viscosity at 25° C. measured on a rotational viscometer (RE550 type viscometer, manufactured by Toki Sangyo Co., Ltd., rotor: 1° 34′×R24) is, for example, preferably 0.1 to 1000 mPa·s, more preferably 0.5 to 100 mPa·s, and further preferably 1 to 50 mPa·s.

The ophthalmic composition according to this embodiment may contain an appropriate amount of various pharmacologically active components or physiologically active components in combination other than the above components as long as the effects of the present invention are not impeded. Such components are not particularly limited, and examples thereof include active ingredients in various medicaments described in The Standards for Marketing Approval of Nonprescription Drugs 2012 (supervised by Society for Regulatory Science of Medical Products). Specific examples of components used in ophthalmic pharmaceuticals are as follows.

Anti-allergic agent: cromoglicates, amlexanox, ibudilast, suplatast, pemirolast potassium, tranilast, olopatadine hydrochloride, levocabastine hydrochloride and acitazanolast and the like.
Antihistamine: chlorpheniramine maleate, diphenhydramine hydrochloride and ketotifen fumarate and the like.
Vasoconstrictor (decongestant): tetrahydrozoline hydrochloride, tetrahydrozoline nitrate, naphazoline hydrochloride, naphazoline nitrate, epinephrine, epinephrine hydrochloride, ephedrine hydrochloride, phenylephrine hydrochloride and dl-methylephedrine hydrochloride and the like.
Anti-inflammatory agent: pranoprofen, glycyrrhizic acid, allantoin, berberine sulfate, berberine chloride, azulene sulfonate, ε-aminocaproic acid, zinc sulfate, zinc lactate, lysozyme, salicylic acid, tranexamic acid, licorice and their salts and the like.
Ocular muscle regulating agents: for example, cholinesterase inhibitors having active centers similar to acetylcholine, specifically neostigmine methyl sulfate, tropicamide, helenien and atropine sulfate and the like.
Disinfectants: acrinol, cetyl pyridinium, benzalkonium chloride, benzethonium chloride, chlorhexidine, polyhexamethylene biguanide and alkyldiaminoethylglycine hydrochloride and the like.
Amino acids: glycine, alanine, γ-aminobutyric acid, aspartic acid, potassium L-aspartate, glutamic acid, arginine, lysine, aminoethylsulfonic acid (taurine), chondroitin sulfate, sodium chondroitin sulfate, sodium hyaluronate and alginic acid and the like.
Vitamins: vitamin B₁, vitamin B₂ (flavin adenine dinucleotide sodium), niacin (nicotinic acid and nicotinic acid amide), pantothenic acid, panthenol, vitamin B₆ (pyridoxine, pyridoxal and pyridoxamine), biotin, folic acid, vitamin B₁₂ (cyanocobalamin, hydroxocobalamin, methylcobalamin and adenosylcobalamin), retinol acetate, retinol palmitate and tocopherol acetate and the like.
Others: for example, sulfamethoxazole, sulfamethoxazole sodium and the like.

The ophthalmic composition according to this embodiment may contain an appropriate amount of one or two or more in combination of various excipients selected as appropriate depending on the application of product form according to a conventional method without impairing the effects of the present invention. Examples of these excipients include various excipients described in Japanese Pharmaceutical Excipients Directory 2007 (edited by International Pharmaceutical Excipients Council Japan). Examples of representative ingredients are the following excipients.

Carriers: for example, aqueous carriers such as water, hydrous ethanol.
Saccharides: for example, glucose, cyclodextrin and the like.
Sugar alcohols: for example, xylitol, sorbitol, mannitol and the like. These may be any of d-isomers, 1-isomers and dl-isomers.
Nonionic surfactants: POE sorbitan fatty acid esters such as POE (20) sorbitan monolaurate (polysorbate 20), POE (20) sorbitan monopalmitate (polysorbate 40), POE (20) sorbitan monostearate (polysorbate 60) and POE (20) sorbitan tristearate (polysorbate 65); POE hydrogenated castor oils such as POE (40) hydrogenated castor oil (polyoxyethylene hydrogenated castor oil 40) and POE (60) hydrogenated castor oil (polyoxyethylene hydrogenated castor oil 60); POE castor oils such as POE (10) castor oil (polyoxyethylene castor oil 10) and POE (35) castor oil (polyoxyethylene castor oil 35); POE alkyl ethers such as POE (9) lauryl ether; POE-POP alkyl ethers such as POE (20) POP (4) cetyl ether; polyoxyethylene-polyoxypropylene block copolymers such as POE (54) POP (39) glycol, POE (120) POP (40) glycol, POE (160) POP (30) glycol, POE (196) POP (67) glycol (poloxamer 407, Pluronic F127) and POE (200) POP (70) glycol; polyethylene glycol monostearate such as polyoxyl 40 stearate, and the like. Note that in the above listed compounds, numerals in parentheses represent number of moles added.
Amphoteric surfactants: N-[2-[[2-(alkylamino)ethyl]amino]ethyl]glycine and salts thereof (also referred to as alkyldiaminoethylglycine or alkylpolyaminoethylglycine) and the like.
Anionic surfactants: alkyl benzene sulfonate, alkyl sulfate, polyoxyethylene alkyl sulfate, α-sulfo fatty acid ester salt, α-olefin sulfonic acid and the like.
Cationic surfactants: benzalkonium chloride, benzethonium chloride and the like.
Antiseptics, disinfectants or antibacterial agents: for example, zinc chloride, alkyldiaminoethylglycine hydrochloride, sodium benzoate, ethanol, benzalkonium chloride, benzethonium chloride, chlorhexidine gluconate, chlorobutanol, sorbic acid, potassium sorbate, sodium dehydro acetate, methyl parahydroxybenzoate, ethyl parahydroxybenzoate, propyl parahydroxybenzoate, butyl parahydroxybenzoate, oxyquinoline sulfate, phenethyl alcohol, benzyl alcohol, biguanide compounds (specifically, polyhexamethylene biguanide, polyhexanide hydrochloride and the like), Glokill (manufactured by Rhodia, trade name) and the like.
Stabilizer: trometamol, sodium formaldehyde sulfoxylate (Rongalite), tocopherol, sodium pyrosulfite, monoethanolamine, aluminum monostearate, glycerin monostearate, dibutylhydroxytoluene, sodium edetate and the like.
Base: for example, octyldodecanol, titanium oxide, potassium bromide, paraffin, plastibase, lanolin, propylene glycol and the like.

The ophthalmic composition according to this embodiment is prepared by adding Component (A) and Component (B) described above, and other components as needed to a carrier so that desired contents are obtained. Specifically, for example, the ophthalmic composition can be prepared by dissolving or suspending the above components in purified water, adjusting the resultant to a predetermined pH and osmotic pressure, and sterilizing the resultant by filtration sterilization or the like.

The water content in the ophthalmic composition according to this embodiment is, based on the total amount of the ophthalmic composition, preferably 85 w/v % or more, more preferably 90 w/v % or more, further preferably 92 w/v % or more, still more preferably 94 w/v % or more, and particularly preferably 96 w/v % or more. As water used in the ophthalmic composition, medicinally, pharmacologically (pharmaceutically) or physiologically acceptable water may be used, and examples of such water include, specifically, distilled water, water, purified water, sterile purified water, water for injection, and distilled water for injection.

The ophthalmic composition according to this embodiment is provided in any container. The container for containing the ophthalmic composition is not particularly limited, and for example, the container may be made of glass, or plastic. Preferably, the container is made of plastic. Examples of plastic include polyethylene terephthalate, polyarylate, polyethylene naphthalate, polycarbonate, polyethylene, polytetrafluoroethylene, polypropylene, polybutylene terephthalate, polyimide, polymethylpentene and copolymers of monomers which consist thereof, and a combination of 2 or more including these materials. Preferred is polyethylene terephthalate. The container for containing the ophthalmic composition may be a transparent container whose inside is visible, or may be an opaque container whose inside is hardly visible. Preferred is a transparent container. Here, “transparent containers” include both colorless transparent containers and colored transparent containers. The ophthalmic composition may be contained in, for example, a colored transparent plastic container or the like in a repeatedly usable multi-dose form, or in a single-use form, and used.

The ophthalmic composition according to this embodiment can be used as a formulation of a pharmaceutical product or quasi-drug, and examples thereof include so-called eye drops [note that eye drops include eye drops which can be instilled during contact lens wear], and in addition, artificial tears, eye washes [note that eye washes include eye washes capable of washing eyes during contact lens wear], compositions for contact lenses [contact lens wetting solutions, contact lens care compositions (contact lens disinfectant solutions, contact lens storage solutions, contact lens cleaning solutions, contact lens cleaning and storage solutions) and the like]. Preferably examples of the present invention include eye drops, artificial tears, eye washes, contact lens wetting solutions, and particularly preferable examples include eye drops, and artificial tears. When the ophthalmic composition is used as a composition for contact lenses, it can be applied to any contact lenses including hard contact lenses, and soft contact lenses. It can also be applied to the contact lenses during wearing. The ophthalmic composition according to this embodiment is suitably applied to contact lenses in view of the effects of inhibition on adsorption of foreign bodies to the cornea due to pressure by wearing contact lenses, or the like, or alleviation of a foreign body sensation in the eye.

The ophthalmic composition can be applied to any contact lenses as long as they are known contact lenses. The ophthalmic composition is particularly preferably applied to soft contact lenses in view of large lens diameter, and large contact area with the eye. Soft contact lenses (SCL) are classified by methods described in, for example, ISO 18369-1:2006 and ISO 18369-1:AMENDMENT1.

The ophthalmic composition according to this embodiment can not only alleviate a subjective symptom itself, but also suppress adsorption of foreign bodies to the cornea in order to alleviate a foreign body sensation in the eye. A foreign body sensation means a subjective symptom of a gritty or stinging eye (a sense of strangeness, irritation). The foreign body sensation may be caused by dryness of the eye, the use of contact lenses, or allergic conjunctivitis. The foreign body sensation is further augmented when accompanied by inflammation. The ophthalmic composition according to this embodiment can be applied with the purpose of prevention or treatment of various symptoms accompanied by a foreign body sensation. The ophthalmic composition is suitable for foreign body sensations caused by allergic conjunctivitis, contact lens wear, dryness of the eye, etc., in view of its capability of inhibiting adsorption of foreign bodies. That is, the ophthalmic composition is suitably used as a foreign body sensation reliever. In other words, the foreign body sensation reliever comprises the ophthalmic composition.

(Method for Imparting an Action of Relieving a Foreign Body Sensation)

In another embodiment of the present invention, there is provided a method for imparting an action of relieving a foreign body sensation in the eye to an ophthalmic composition, comprising incorporating (A) polysorbate 80 and (B) a refreshing agent in the ophthalmic composition,

wherein a polydispersity of the polysorbate 80 in the ophthalmic composition is 1.00 to 1.26, and

the refreshing agent comprises at least one selected from the group consisting of camphor, bomeol and geraniol.

In still another embodiment of the present invention, there is provided a method for imparting an action of relieving a foreign body sensation in the eye to an ophthalmic composition by incorporating (A) polysorbate 80 and (B) a refreshing agent therein,

wherein a polydispersity of the polysorbate 80 in the ophthalmic composition is 1.00 to 1.26.

(Method for Relieving a Foreign Body Sensation in the Eye)

In still another embodiment of the present invention, there is provided a method for relieving a foreign body sensation in the eye, comprising a step of contacting an ophthalmic composition comprising polysorbate 80 and a refreshing agent with an eye or a contact lens of a subject,

wherein a polydispersity of the polysorbate 80 in the ophthalmic composition is 1.00 to 1.26. Examples of the subject include mammals such as human.

According to the ophthalmic composition according to this embodiment, the combination of the polysorbate 80 having a polydispersity of 1.00 to 1.26 in the ophthalmic composition, and a refreshing agent suppresses adsorption of foreign bodies to the cornea, enabling more alleviation of a foreign body sensation in the eye.

(Production Method)

In the production of the ophthalmic composition according to this embodiment, it is suggested that the incorporation of the polysorbate 80 having a polydispersity of 1.00 to 1.26 and a refreshing agent in the ophthalmic composition can contribute to the provision of a formulation having a sustained or improved inhibitory effect (inhibitory action) on adsorption of foreign bodies to the cornea.

### EXAMPLES

The present invention will be described in more detail based on Examples below, but the present invention is not limited by these Examples.

### Test Example 1

### Measurement on the Amount of Foreign Bodies Adsorbed to Corneal Epithelial Cells

### (1) Test Method

Polysorbate 80 having different polydispersities described in Table 1 were used to prepare test solutions A and test solutions B described in Table 2 and Table 3, and the polydispersity of the polysorbate 80 in each test solution was measured. Subsequently, each test solution with fluorescently labeled albumin (final concentration 0.2%) added thereto was added to a culture medium, and the effect on adsorption of foreign bodies to corneal epithelial cells was evaluated.

The human corneal epithelial cell line HCE-T (Riken BioResource Center, No. RCB2280) at 3.0×10⁵ cells/well was seeded in 600 μL of a culture medium per well of a 48-well microplate (manufactured by Corning Inc.), and cultured at a condition of 37° C., 5% CO₂ for 24 hours. After the culture, the culture medium was suction removed, and 200 μL of each test solution with albumin fluorescently labeled with fluorescein isothiocyanate (FITC) (final concentration 0.2%) added thereto was put therein, and allowed to stand still at room temperature for 60 minutes. After the treatment, the solution in the well was suction removed, and 600 μL of phosphate buffer was added to each well. After the solution in the well was suction removed for cleaning, 200 μL of phosphate buffer was put therein, and the fluorescence value in each well was measured using a fluorescence plate reader (Fluoroskan Ascent CF, manufactured by MTX Lab Systems, Inc.) at an excitation wavelength of 495 nm/an emission wavelength of 520 nm. The obtained fluorescence value was converted into the amount of foreign bodies (the amount of albumin) based on the calibration curve of the amount of foreign bodies and the fluorescence value prepared beforehand. From the obtained amount of foreign bodies, the inhibition percentage (%) of adsorption of foreign bodies was calculated based on Expression (1). Then, an increase or decrease in the inhibition percentage of adsorption of foreign bodies was calculated based on Expression (2). In Expression (1), Control refers to a culture medium which does not contain Component (A) or Component (B).

Inhibition percentage (%) of adsorption of foreign bodies=[1−{(the amount of foreign bodies in each test solution)/(the amount of foreign bodies in Control)}]×100 Expression (1)

Increase or decrease in the inhibition percentage of adsorption of foreign bodies={the inhibition percentage (%) of adsorption of foreign bodies when using the test solution B}−{the inhibition percentage (%) of adsorption of foreign bodies when using the test solution A} Expression (2)

### (2) Test Results

The results are shown in Table 4. It is shown that when combining various Components (A) having different polydispersities and a Component (B), in Components (A) having polydispersities of 1.28 or more in the test solutions, the inhibitory effect on adsorption of foreign bodies is reduced (Comparative Examples 1 to 2), but in Components (A) having polydispersities of less than 1.28 in the test solution, the inhibitory effect on adsorption of foreign bodies is increased (Examples 1 to 4).

TABLE 1

| | | | Z-average |
|---|---|---|---|
| | | Weight average | molecular |
| | Polydispersity | molecular weight | weight |
| Polysorbate 80 (No. 1) | 1.34 | 1978 | 2641 |
| Polysorbate 80 (No. 2) | 1.28 | 1781 | 2276 |
| Polysorbate 80 (No. 3) | 1.23 | 1971 | 2424 |
| Polysorbate 80 (No. 4) | 1.21 | 1731 | 2090 |
| Polysorbate 80 (No. 5) | 1.19 | 1837 | 2189 |
| Polysorbate 80 (No. 6) | 1.17 | 1681 | 1971 |

TABLE 2

| | Comparative | Comparative | | | | |
|---|---|---|---|---|---|---|
| | Example | Example | Example | Example | Example | Example |
| Test solution A | 1-A | 2-A | 1-A | 2-A | 3-A | 4-A |
| (A) Polysorbate 80 (No. 1) | 0.01 | — | — | — | — | — |
| (A) Polysorbate 80 (No. 2) | — | 0.01 | — | — | — | — |
| (A) Polysorbate 80 (No. 3) | — | — | 0.01 | — | — | — |
| (A) Polysorbate 80 (No. 4) | — | — | — | 0.01 | — | — |
| (A) Polysorbate 80 (No. 5) | — | — | — | — | 0.01 | — |
| (A) Polysorbate 80 (No. 6) | — | — | — | — | — | 0.01 |
| Purified water | Balance | Balance | Balance | Balance | Balance | Balance |
| Total amount | 100 mL | 100 mL | 100 mL | 100 mL | 100 mL | 100 mL |
| Polydispersity of the | 1.34 | 1.28 | 1.23 | 1.21 | 1.19 | 1.17 |
| polysorbate 80 in the test | | | | | | |
| solution | | | | | | |

(Unit: g/100 mL)

TABLE 3

| | Comparative | Comparative | | | | |
|---|---|---|---|---|---|---|
| | Example | Example | Example | Example | Example | Example |
| Test solution B | 1-B | 2-B | 1-B | 2-B | 3-B | 4-B |
| (A) Polysorbate 80 (No. 1) | 0.01 | — | — | — | — | — |
| (A) Polysorbate 80 (No. 2) | — | 0.01 | — | — | — | — |
| (A) Polysorbate 80 (No. 3) | — | — | 0.01 | — | — | — |
| (A) Polysorbate 80 (No. 4) | — | — | — | 0.01 | — | — |
| (A) Polysorbate 80 (No. 5) | — | — | — | — | 0.01 | — |
| (A) Polysorbate 80 (No. 6) | — | — | — | — | — | 0.01 |
| (B) I-Menthol | 0.001 | 0.001 | 0.001 | 0.001 | 0.001 | 0.001 |
| Purified water | Balance | Balance | Balance | Balance | Balance | Balance |
| Total amount | 100 mL | 100 mL | 100 mL | 100 mL | 100 mL | 100 mL |
| Polydispersity of the | 1.34 | 1.28 | 1.23 | 1.21 | 1.19 | 1.17 |
| polysorbate 80 in the test | | | | | | |
| solution | | | | | | |

(Unit: g/100 mL)

TABLE 4

| | Comparative | Comparative | | | | |
|---|---|---|---|---|---|---|
| | Example 1 | Example 2 | Example 1 | Example 2 | Example 3 | Example 4 |
| Increase or decrease | −33.4 | −5.9 | 2.1 | 4.5 | 8.6 | 21.1 |
| in the inhibition | | | | | | |
| percentage of | | | | | | |
| adsorption of foreign | | | | | | |
| bodies | | | | | | |
| Polydispersity of the | 1.34 | 1.28 | 1.23 | 1.21 | 1.19 | 1.17 |
| polysorbate 80 in the | | | | | | |
| test solution | | | | | | |

### Test Example 2

### Sensory Test: Effect of Alleviating a Foreign Body Sensation

### (1) Test Method

Each eye drops was prepared according to Table 5 below following a conventional method, and the polydispersity of the polysorbate 80 in each eye drops was measured. Each eye drops was filled in a container made of polyethylene terephthalate. To subjects usually conscious of a foreign body sensation in the eye, eye drops containing (A) polysorbate 80 and (B) 1-menthol, where the polydispersity of the polysorbate 80 in the eye drops was less than 1.21 (Example 5 to 7), was administered singly to the left eye, and eye drops containing (A) polysorbate 80 and (B) 1-menthol, where the polydispersity of the polysorbate 80 in the eye drops was 1.34 or more (Comparative Example 3), was administered singly to the right eye. Subsequently, using a 10-cm line scaled from 0 to 100 shown in Table 6, a foreign body sensation in the eye after instilling the eye drops was evaluated by a Visual Analog Scale method (VAS method). The evaluation of a foreign body sensation was separately conducted in the case of 5 subjects of non-contact lens wearers, and in the case of 3 subjects of contact lens wearers.

### (2) Test Results

The results are shown in Table 5. It was confirmed that when the eye drops of Examples 5 to 7 containing (A) polysorbate 80 and (B) 1-menthol, where the polydispersity of the polysorbate 80 in the eye drops was less than 1.34, were used, a significant effect of alleviating a foreign body sensation was shown.

TABLE 5

| | Comparative | | | |
|---|---|---|---|---|
| | Example 3 | Example 5 | Example 6 | Example 7 |
| (A) Polysorbate 80 (No. 1) | 0.01 | — | — | — |
| (A) Polysorbate 80 (No. 4) | — | 0.01 | — | — |
| (A) Polysorbate 80 (No. 5) | — | — | 0.01 | — |
| (A) Polysorbate 80 (No. 6) | — | — | — | 0.01 |
| (B) I-Menthol | 0.001 | 0.001 | 0.001 | 0.001 |
| Boric acid | 1.8 | 1.8 | 1.8 | 1.8 |
| Borax | 0.35 | 0.35 | 0.35 | 0.35 |
| Sodium edetate | 0.01 | 0.01 | 0.01 | 0.01 |
| Hydrochloric acid | Appropriate | Appropriate | Appropriate | Appropriate |
| | amount | amount | amount | amount |
| Sodium hydroxide | Appropriate | Appropriate | Appropriate | Appropriate |
| | amount | amount | amount | amount |
| Purified water | Balance | Balance | Balance | Balance |
| pH | 7.0 | 7.0 | 7.0 | 7.0 |
| Total amount | 100 mL | 100 mL | 100 mL | 100 mL |
| Polydispersity of the polysorbate 80 in | 1.34 | 1.21 | 1.19 | 1.17 |
| the eye drops | | | | |
| Foreign body sensation | 68.1 | 41.7 | 37.0 | 31.8 |
| (non-contact lens wearer) | | | | |
| Foreign body sensation | 63.2 | 31.8 | 27.8 | 18.0 |
| (contact lens wearer) | | | | |

(Unit: g/100 mL)

TABLE 6

| Scale | Evaluation of a foreign body sensation |
|---|---|
| 0 | No foreign body sensation at all |
| 100 | The strongest foreign body sensation which |
| | has ever been experienced |

### Test Example 3

### Sensory Test: Effect of Improving a Foreign Body Sensation

### (1) Test Method

A foreign body sensation in the eye after instilling eye drops was evaluated by the same method as in Test Example 2, except that each eye drops was prepared according to Table 7 and Table 8 below following a conventional method. In the tests of Comparative Example 4, Example 8 and Example 9, subjects were 5 non-contact lens wearers and 5 contact lens wearers. In other tests, subjects were 3 non-contact lens wearers and 3 contact lens wearers.

### (2) Test Results

The results are shown in Table 7 and Table 8. It was confirmed that when eye drops of Examples 8 to 15 containing (A) polysorbate 80 and (B) a refreshing agent (d-borneol, d-camphor or geraniol), where the polydispersity of the polysorbate 80 in the eye drops was less than 1.34, were used, a significant effect of alleviating a foreign body sensation was shown.

TABLE 7

| | Comparative | | | Comparative | Example | Comparative | Example |
|---|---|---|---|---|---|---|---|
| | Example 4 | Example 8 | Example 9 | Example 5 | 10 | Example 6 | 11 |
| (A) Polysorbate | 0.01 | — | — | 0.3 | — | 0.1 | — |
| 80 (No. 1) | | | | | | | |
| (A) Polysorbate | — | 0.01 | — | — | — | — | — |
| 80 (No. 4) | | | | | | | |
| (A) Polysorbate | — | — | 0.01 | — | 0.3 | — | 0.1 |
| 80 (No. 6) | | | | | | | |
| (B) d-Borneol | 0.001 | 0.001 | 0.001 | 0.05 | 0.05 | 0.005 | 0.001 |
| (B) d-Camphor | — | — | — | — | — | — | — |
| (B) Geraniol | — | — | — | — | — | — | — |
| Boric acid | 1.8 | 1.8 | 1.8 | 1.8 | 1.8 | 1.8 | 1.8 |
| Borax | 0.35 | 0.35 | 0.35 | 0.35 | 0.35 | 0.35 | 0.35 |
| Sodium edetate | 0.01 | 0.01 | 0.01 | 0.01 | 0.01 | 0.01 | 0.01 |
| Hydrochloric | Appropriate | Appropriate | Appropriate | Appropriate | Appropriate | Appropriate | Appropriate |
| acid | amount | amount | amount | amount | amount | amount | amount |
| Sodium | Appropriate | Appropriate | Appropriate | Appropriate | Appropriate | Appropriate | Appropriate |
| hydroxide | amount | amount | amount | amount | amount | amount | amount |
| Purified water | Balance | Balance | Balance | Balance | Balance | Balance | Balance |
| pH | 7.1 | 7.1 | 7.1 | 7.1 | 7.1 | 7.1 | 7.1 |
| Total amount | 100 mL | 100 mL | 100 mL | 100 mL | 100 mL | 100 mL | 100 mL |
| Polydispersity | 1.34 | 1.21 | 1.17 | 1.34 | 1.17 | 1.34 | 1.17 |
| of the | | | | | | | |
| polysorbate 80 | | | | | | | |
| in the eye drops | | | | | | | |
| Foreign body | 50.8 | 28.0 | 27.8 | 35.0 | 16.3 | 40.0 | 20.7 |
| sensation | | | | | | | |
| (non-contact | | | | | | | |
| lens wearer) | | | | | | | |
| Foreign body | 48.2 | 23.0 | 20.6 | 48.0 | 25.5 | 30.0 | 14.0 |
| sensation | | | | | | | |
| (contact lens | | | | | | | |
| wearer) | | | | | | | |

(Unit: g/100 mL)

TABLE 8

| | Comparative | | | Comparative | | |
|---|---|---|---|---|---|---|
| | Example 7 | Example 12 | Example 13 | Example 8 | Example 14 | Example 16 |
| (A) Polysorbate 80 (No. 1) | 0.01 | — | — | 0.01 | — | — |
| (A) Polysorbate 80 (No. 4) | — | 0.01 | — | — | 0.01 | — |
| (A) Polysorbate 80 (No. 6) | — | — | 0.01 | — | — | 0.01 |
| (B) d-Borneol | — | — | — | — | — | — |
| (B) d-Camphor | 0.001 | 0.001 | 0.001 | — | — | — |
| (B) Geraniol | — | — | — | 0.001 | 0.001 | 0.001 |
| Boric acid | 1.8 | 1.8 | 1.8 | 1.8 | 1.8 | 1.8 |
| Borax | 0.35 | 0.35 | 0.35 | 0.35 | 0.35 | 0.35 |
| Sodium edetate | 0.01 | 0.01 | 0.01 | 0.01 | 0.01 | 0.01 |
| Hydrochloric acid | Appropriate | Appropriate | Appropriate | Appropriate | Appropriate | Appropriate |
| | amount | amount | amount | amount | amount | amount |
| Sodium hydroxide | Appropriate | Appropriate | Appropriate | Appropriate | Appropriate | Appropriate |
| | amount | amount | amount | amount | amount | amount |
| Purified water | Balance | Balance | Balance | Balance | Balance | Balance |
| pH | 7.1 | 7.1 | 7.1 | 7.1 | 7.1 | 7.1 |
| Total amount | 100 mL | 100 mL | 100 mL | 100 mL | 100 mL | 100 mL |
| Polydispersity of the | 1.34 | 1.21 | 1.17 | 1.34 | 1.21 | 1.17 |
| polysorbate 80 in the eye | | | | | | |
| drops | | | | | | |
| Foreign body sensation | 36.7 | 19.0 | 8.7 | 55.0 | 37.7 | 21.7 |
| (non-contact lens wearer) | | | | | | |
| Foreign body sensation | 39.7 | 17.0 | 15.7 | 48.7 | 23.3 | 21.3 |
| (contact lens wearer) | | | | | | |

(Unit: g/100 mL)

1. An ophthalmic composition comprising:
polysorbate 80, and
a refreshing agent,
wherein a polydispersity of the polysorbate 80 in the ophthalmic composition is 1.00 to 1.26.

2. The ophthalmic composition according to claim 1, wherein the refreshing agent comprises at least one selected from the group consisting of menthol, camphor, borneol and geraniol.

3. The ophthalmic composition according to claim 1, wherein a total content of the refreshing agent is 0.0001 to 100 parts by mass with respect to 1 part by mass of a content of the polysorbate 80 contained in the ophthalmic composition.

4. A method for imparting an action of relieving a foreign body sensation to an ophthalmic composition by incorporating polysorbate 80 and a refreshing agent therein,
wherein a polydispersity of the polysorbate 80 in the ophthalmic composition is 1.00 to 1.26.

5. The ophthalmic composition according to claim 2, wherein a total content of the refreshing agent is 0.0001 to 100 parts by mass with respect to 1 part by mass of a content of the polysorbate 80 contained in the ophthalmic composition.
